(19) United States
(12) Patent Application Publication
(10) Pub. No.: US 2026/0115441 A1
(43) Pub. Date: Apr. 30, 2026

(54) APPARATUS AND METHOD FOR INTRADERMAL DELIVERY OF ACTIVES FOLLOWING PRE-CONDITIONING OF SKIN

(71) Applicant: VIAS Partners, LLC, Doylestown, PA (US)

(72) Inventor: Sanjay Batra, New Hope, PA (US)

(73) Assignee: VIAS Partners, LLC, Doylestown, PA (US)

(21) Appl. No.: 19/369,601

(22) Filed: Oct. 27, 2025

Related U.S. Application Data
(60) Provisional application No. 63/711,853, filed on Oct. 25, 2024.

(51) Int. Cl. A61M 37/00

(52) U.S. Cl. CPC A61M 37/0015; A61M 2037/0007; A61M 2037/0023; A61M 2037/003; A61M 2037/0061; A61M 2210/04

(57) ABSTRACT

The present disclosure provides a method for intradermal delivery of a serum comprising pre-conditioning a target area of the subject's stratum corneum by contacting the area with a device comprising a cartridge and nanotip, the pre-conditioning comprising moving the device in vertical, horizontal, and circular motions over the target area for 15 to 20 seconds per 2×2 cm portion to mechanically abrade the stratum corneum; filling the cartridge with a serum composition comprising biologically active agents; inserting the cartridge into the device inlet and holding vertically for 5 seconds; applying the serum to the pre-conditioned area by actuating the device to vibrate at 5800 to 8800 revolutions per minute and dispense serum through micro-ports positioned adjacent the nanotip, comprising moving the device in vertical, horizontal, and circular motions for up to 60 seconds; and optionally massaging the serum into the treated area.

### CROSS-REFERENCE TO RELATED APPLICATIONS

This application claims priority to U.S. Application No. 63/711,853, titled APPARATUS AND METHOD FOR INTRADERMAL DELIVERY OF ACTIVES FOLLOWING PRE-CONDITIONING OF SKIN, filed Oct. 25, 2024, which is hereby incorporated by reference in its entirety.

### FIELD OF INVENTION

The present disclosure relates to methods and apparatuses for intradermal delivery of therapeutic compounds, and more particularly to minimally invasive procedures for delivering actives to skin following pre-conditioning to enhance permeability and treat skin conditions and diseases.

### BACKGROUND

Topical delivery of therapeutic compounds to skin faces substantial challenges due to the natural barrier function of the epidermis. The outermost layer of skin, known as the stratum corneum, typically measures between 3000 and 5000 micrometers in thickness and serves as a protective barrier against environmental factors. This same protective function that shields the body from external threats also impedes the penetration of beneficial therapeutic compounds applied to the skin surface.

Various approaches have been explored to enhance transdermal delivery of therapeutic compounds. These methods include the application of ultrasound energy, electrical energy, and mechanical energy to temporarily disrupt the epidermal barrier. However, such techniques have demonstrated limited effectiveness in achieving adequate penetration of therapeutic compounds through the skin barrier to reach deeper tissue layers where they can provide therapeutic benefit.

The challenge of effective transdermal delivery is particularly pronounced when treating skin conditions and diseases that affect deeper layers of the epidermis. Many therapeutic compounds, including peptides, growth factors, and other bioactive molecules, have molecular sizes or properties that prevent them from readily crossing the stratum corneum barrier. This limitation reduces the therapeutic efficacy of topically applied treatments and may require higher concentrations or more frequent applications to achieve desired outcomes.

Current methods for enhancing skin permeability often involve complex procedures or may cause discomfort to patients. Additionally, many existing approaches fail to provide controlled and predictable enhancement of skin permeability, leading to inconsistent therapeutic results. The timing and coordination of barrier disruption with therapeutic compound application also presents challenges in existing methodologies.

There remains a need for improved methods and devices that can effectively and safely enhance the delivery of therapeutic compounds through the skin barrier. Such improvements would benefit the treatment of various skin conditions and diseases by enabling more effective penetration of therapeutic compounds to target tissue layers while maintaining patient comfort and safety.

### SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to a method for intradermal delivery of a biological to a subject. The method comprises pre-conditioning a target area of the subject's stratum corneum by contacting the area with a device comprising a cartridge and a nanotip. The pre-conditioning comprises moving the device in vertical, horizontal, and circular motions over the target area for about 15 to 20 seconds per 2×2 cm portion of the area to mechanically abrade the stratum corneum. The method comprises filling a cartridge of the device with a serum composition comprising one or more biologically active agents. The method comprises inserting the cartridge into an inlet of the device and holding the device vertically for about 5 seconds. The method comprises applying the serum to the pre-conditioned area by actuating the device to vibrate at a speed of from about 5800 to about 8800 revolutions per minute (RPM) and dispense the serum through a plurality of micro-ports positioned adjacent the nanotip. The applying comprises moving the device in vertical, horizontal, and circular motions for up to about 60 seconds. The method comprises optionally massaging the serum into the treated area.

The foregoing general description of the illustrative embodiments and the following detailed description thereof are merely exemplary aspects of the teachings of this disclosure and are not restrictive.

### BRIEF DESCRIPTION OF FIGURES

The features, and advantages of the invention will be apparent from the following more detailed description of certain embodiments of the invention and as illustrated in the accompanying drawings in which:

FIG. 1 illustrates a schematic representation of exemplary methods and apparatuses for delivery of actives to a subject, according to aspects of the present disclosure;

FIG. 2 illustrates a perspective view of a device with a body and a head, according to aspects of the present disclosure;

FIG. 3 illustrates a perspective view of the head of the device of FIG. 2 with a nanochip having tips, according to aspects of the present disclosure;

FIG. 4 illustrates a perspective view of the device of FIG. 2 and a cartridge configured for positioning within the body, according to aspects of the present disclosure;

FIG. 5 illustrates a flow diagram of a method for delivery of actives to a subject, according to aspects of the present disclosure; and

FIG. 6 illustrates high resolution ultrasonic images showing views of a human scalp before conditioning, after conditioning, and after self-repair, according to aspects of the present disclosure.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to and can be employed in other apparatuses and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “at least” prior to a number or series of numbers (e.g., “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range. Ranges provided herein are understood to include all individual integer values and all subranges within the ranges.

All references cited herein are hereby incorporated by reference in their entireties. In the event of a conflict in a definition in the present disclosure and that of a cited reference, the present disclosure controls.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care films and/or the oral care compositions of the instant disclosure can be free or essentially free of all components and elements positively recited throughout the instant disclosure. In some instances, the oral care films and/or oral care compositions of the present disclosure may be substantially free of non-incidental amounts of the ingredient(s) or compound(s) described herein. A non-incidental amount of an ingredient or compound is the amount of that ingredient or compound that is added into the oral care films and/or the oral care composition by itself. For example, an oral care film and/or an oral care composition may be substantially free of a non-incidental amount of an ingredient or compound, although such ingredient(s) or compound(s) may be present as part of a raw material that is included as a blend of two or more compounds.

As used herein, the term “topical serum” refers to any formulation that may be applied directly to a subject's skin to deliver biologically active agents, or “actives”, through the subject's skin. As used herein, the term “intradermal delivery” refers to the delivery of actives into, within, or through the subject's skin, such as after application of a topical serum.

One skilled in the art will understand that a subject's skin includes multiple layers, including the epidermis, which is the outermost layer of skin. The epidermis is understood to have sublayers, including (listed in order from the outermost sublayer to the innermost sublayer), the stratum corneum, stratum lucideum, stratum granulosum, stratum spinosum, and stratum basale. The terms “skin” and “epidermis” may be used interchangeably herein. Accordingly, it is understood that application of a topical serum to a subject's skin may include application of the serum to the epidermis and delivery of the serum to the stratum corneum.

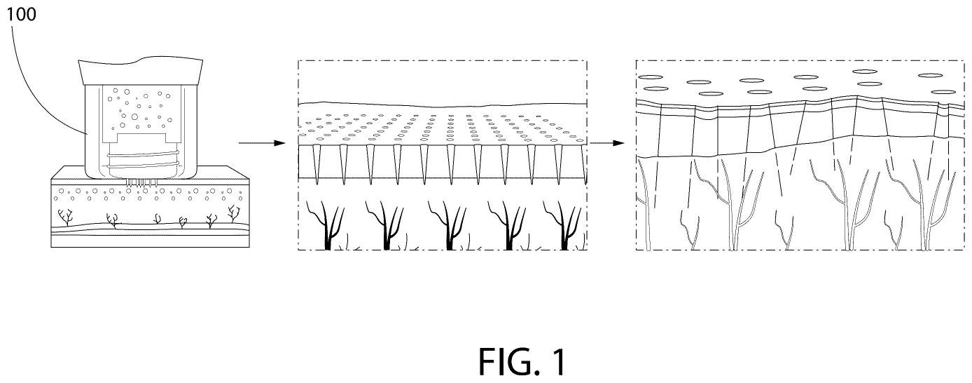

Referring to FIGS. 1-6, embodiments of the present disclosure relate to methods and apparatuses for intradermal delivery of therapeutic compounds to subjects for treating various skin conditions and diseases.

The skin's natural barrier function, particularly the stratum corneum layer, presents a challenge for effective transdermal delivery of biologically active agents. The stratum corneum typically measures between 3000 and 5000 micrometers in thickness and acts as a physical barrier that impedes the penetration of topically applied therapeutic compounds.

Conventional approaches for enhancing transdermal delivery, including ultrasound energy, electrical energy, and mechanical energy, have demonstrated limited effectiveness in overcoming the barrier properties of the stratum corneum. These methods often fail to provide adequate penetration of therapeutic compounds to deeper layers of the epidermis where treatment may be most beneficial.

With continued reference to FIG. 1, the present disclosure addresses these limitations through a method that involves pre-conditioning the skin prior to application and delivery of therapeutic compounds utilizing a delivery device 100. The pre-conditioning process temporarily disrupts or abrades the stratum corneum, thereby exposing deeper layers of the epidermis and enhancing the permeability of the skin barrier. This temporary disruption creates a window of opportunity for improved delivery of therapeutic compounds to sublayers of the epidermis, including the stratum lucideum, stratum granulosum, stratum spinosum, and stratum basale. Without this pre-conditioning step, topical absorption may be paradoxically impeded, as the serum itself can form an occlusive film that reinforces the skin's barrier properties rather than promoting penetration. The pre-conditioning phase therefore mitigates this effect by first increasing surface permeability, allowing subsequent serum application to achieve meaningful intradermal delivery.

The therapeutic compounds delivered through this system include various biologically active agents including, but not limited to, peptides, growth factors, platelet-rich plasma, exosomes, secretomes, vitamins, botanicals, drugs such as minoxidil or finasteride, and combinations thereof. These compounds may be formulated in topical serum compositions that can be applied to the pre-conditioned skin area. The serum compositions may be autologous, derived from the same subject receiving treatment, or allogeneic, derived from a different subject.

The pre-conditioning approach provides enhanced absorption of therapeutic compounds compared to conventional topical application methods. The temporary nature of the stratum corneum disruption allows for improved compound penetration while the skin's natural healing processes restore the barrier function within a relatively short timeframe, typically within 30 minutes or less following the pre-conditioning procedure.

The pre-conditioning methodology described herein provides unexpected and advantageous results in the delivery of therapeutic compounds to skin tissue. The mechanical abrasion of the stratum corneum through the pre-conditioning process achieves temporary removal or substantial removal of this outermost barrier layer, exposing deeper epidermal layers that are normally inaccessible to topically applied compounds. This temporary disruption removes at least 75%, at least 80%, at least 90%, at least 95%, or at least 99% of the stratum corneum thickness, creating enhanced permeability pathways for therapeutic compound penetration.

The enhanced permeability achieved through pre-conditioning enables improved absorption of various biologically active agents including peptides, growth factors, exosomes, and secretomes into deeper tissue layers. These therapeutic compounds penetrate beyond the stratum corneum to reach the stratum lucideum, stratum granulosum, stratum spinosum, and stratum basale, where the compounds can exert their therapeutic effects more effectively than through conventional topical application methods.

The pre-conditioning approach provides a synergistic effect when combined with the subsequent delivery procedure. The initial mechanical disruption of the stratum corneum creates microchannels and enhanced permeability pathways that facilitate the penetration of therapeutic compounds during the delivery phase. This two-step process achieves greater compound absorption compared to single-step delivery methods that attempt to penetrate the intact stratum corneum barrier.

The temporary nature of the stratum corneum disruption represents an advantageous aspect of the pre-conditioning methodology. The skin's natural healing processes restores the barrier function through rapid self-repair mechanisms, typically within 10 to 15 minutes following the pre-conditioning procedure. This rapid restoration minimizes the risk of prolonged barrier compromise while providing sufficient time for therapeutic compound delivery during the treatment window.

Hair follicles extending through the epidermis may serve as additional conduits for therapeutic compound delivery following pre-conditioning. The disruption of the stratum corneum around hair follicle openings enables therapeutic compounds to attach to or position themselves within the follicular structures, facilitating delivery to deeper epidermal and dermal layers through these natural pathways.

The pre-conditioning methodology achieves enhanced bioavailability of therapeutic compounds at the target tissue sites. By bypassing the barrier function of the intact stratum corneum, the therapeutic compounds reach their intended sites of action in higher concentrations and with greater consistency compared to conventional topical delivery methods. This enhanced bioavailability translates to improved therapeutic outcomes for various skin conditions and diseases.

The controlled nature of the pre-conditioning process provides predictable and reproducible results across different treatment sessions and subjects. The mechanical parameters of the pre-conditioning procedure, including the depth of stratum corneum disruption and the duration of barrier compromise, may be controlled to optimize therapeutic compound delivery while minimizing potential adverse effects associated with excessive barrier disruption.

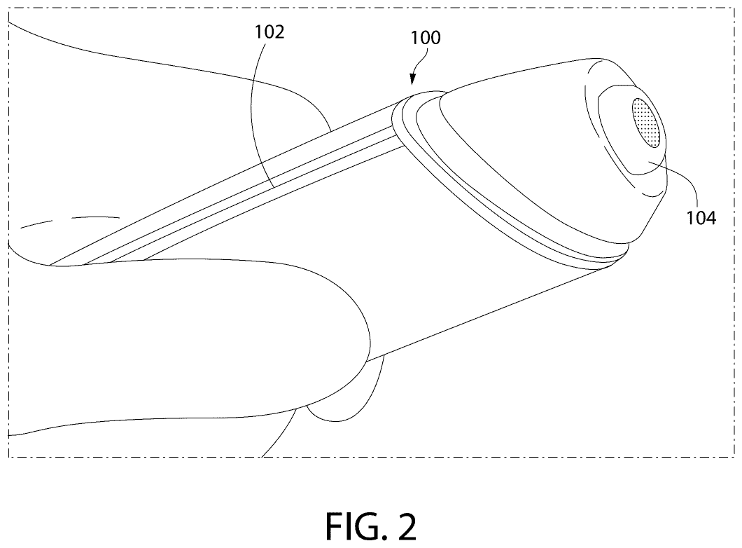

Referring to FIG. 2, the delivery device 100 is configured to perform the pre-conditioning and biological active delivery procedures described herein. The device 100 includes a body 102 and a head 104 that together form the complete treatment apparatus. The body 102 extends longitudinally and is configured to be held by a practitioner during treatment procedures.

The body 102 has a first end and a second end, where the first end forms the terminal end of the device 100. The second end of the body 102 is configured to be coupled or removably coupled to the head 104. The coupling between the body 102 and the head 104 may be achieved through various connection methods including threading, snap connections, press fit connections, or other suitable coupling mechanisms that provide secure attachment during operation.

As shown in FIG. 2, the body 102 is hollow and serves as a housing for additional components of the device 100. The hollow internal structure of the body 102 accommodates various operational components such as vibration mechanisms, control electronics, power sources, and other elements that enable the device 100 to perform the pre-conditioning and delivery functions. The hollow configuration also provides space for cartridge insertion and serum delivery pathways.

The body 102 forms an ergonomic shape designed to fit comfortably within a practitioner's hands or fingers during treatment procedures. The ergonomic design facilitates proper grip and control of the device 100 during the various motions required for pre-conditioning and serum delivery, including vertical, horizontal, and circular movements over the treatment area. The ergonomic configuration reduces practitioner fatigue and enhances treatment precision during extended treatment sessions.

With continued reference to FIG. 2, the head 104 has a first end and a second end, where the first end is configured to be coupled or removably coupled to the body 102. The second end of the head 104 forms an end of the device 100 and includes a planar terminus end surface. The end surface is flat or substantially flat and is configured to interact with a subject's skin during treatment procedures.

The planar terminus end surface of the head 104 provides a stable interface for contact with the skin surface during both pre-conditioning and delivery procedures. The flat or substantially flat configuration enables consistent contact pressure and uniform treatment coverage across the target area. The planar surface also facilitates proper positioning of treatment components such as nanotips and micro-ports for effective therapeutic compound delivery.

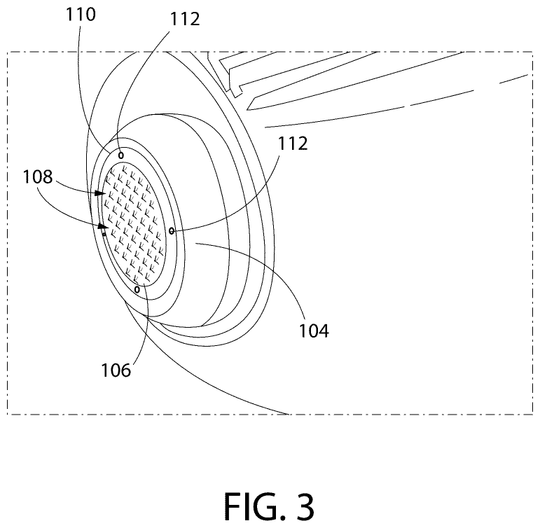

Referring to FIG. 3, the head 104 includes a nanochip 106 positioned at an outer edge 110 of the head 104. The nanochip 106 is positioned at or on the end surface of the head 104 to provide the interface for skin contact during treatment procedures. The positioning of the nanochip 106 at the outer edge 110 enables direct contact with the skin surface while maintaining proper alignment with other components of the device 100.

The nanochip 106 includes a plurality of tips 108 extending from the surface of the nanochip 106. As shown in FIG. 3, the tips 108 extend perpendicularly or substantially perpendicularly from the end surface of the head 104. The perpendicular extension of the tips 108 provides consistent penetration depth and uniform treatment coverage across the target skin area during both pre-conditioning and delivery procedures.

The tips 108 are integrally formed with the nanochip 106, such that the nanochip 106 and the tips 108 are made from the same material. In some cases, the material may be monocrystalline silicon, such as monocrystalline silicon with a purity of 99.999%. The integral formation of the tips 108 with the nanochip 106 provides structural integrity and consistent performance characteristics across all treatment elements.

The height of the tips 108 is between 0.12 and 1.0 mm, with the perpendicular extension of the tips 108 measured from the end surface of the head 104. In some cases, the tips 108 may have a height between 0.12 and 0.3 mm. The controlled height range of the tips 108 enables precise penetration depth control to achieve stratum corneum disruption without penetrating through the stratum corneum layer into deeper epidermal sublayers.

All of the plurality of tips 108 may be of the same height to provide uniform penetration depth across the treatment area. In some cases, the heights of the tips 108 may differ to create varied penetration patterns or to accommodate different treatment requirements. The uniform or varied height configurations may be selected based on the specific therapeutic goals and the characteristics of the target skin area.

With continued reference to FIG. 3, the tips 108 may be hollow or non-hollow depending on the specific treatment requirements and serum delivery mechanisms. In some cases, all of the plurality of tips 108 may be hollow to enable serum delivery through the tip structures. In other cases, all of the plurality of tips 108 may be non-hollow when serum delivery occurs through alternative pathways such as micro-ports positioned adjacent to the nanochip 106.

The tips 108 form an array of tips arranged in various geometric configurations on the nanochip 106. The array may be in any shape or combination of shapes, and may be continuous or discontinuous across the surface of the nanochip 106. The array configurations may include circles, triangles, rectangles, squares, rhomboids, trapezoids, and other regular or irregular polygons. The geometric arrangement of the tips 108 is selected to optimize treatment coverage and therapeutic compound delivery efficiency.

The array of tips 108 includes between 6 and 288 tips total, depending on the size of the nanochip 106 and the desired treatment density. The number of tips 108 in the array is adjusted to provide appropriate treatment intensity while maintaining controlled penetration characteristics. The tip density within the array influences the degree of stratum corneum disruption and the resulting enhancement of therapeutic compound permeability.

In the illustrated embodiment, the head 104 further includes one or more micro-ports 112 configured for controlled dispensing of therapeutic serum compositions during treatment procedures. The micro-ports 112 are positioned circumferentially around the nanochip 106 at predetermined radial distances to optimize serum distribution patterns across the treatment area. The micro-ports 112 have port diameters ranging from about 0.05 mm to about 1.0 mm, with specific configurations including diameters of 0.1 mm, 0.2 mm, and 0.5 mm. In the illustrated embodiment, the head 104 includes four micro-ports. In some embodiments, the head 104 may include between 2 and 8 micro-ports. The micro-ports 112 are positioned at angular intervals of about 45 degrees to about 180 degrees around the nanochip 106, depending on the total number of ports employed. Each micro-port 112 is fluidly connected to the cartridge 114 through internal flow channels within the head 104, enabling controlled serum delivery with flow rates ranging from about 0.01 mL/min to about 2.0 mL/min per port. The micro-ports 112 may be manufactured using precision drilling, laser ablation, or micro-machining techniques to achieve the desired dimensional tolerances and surface finish characteristics for optimal serum flow dynamics.

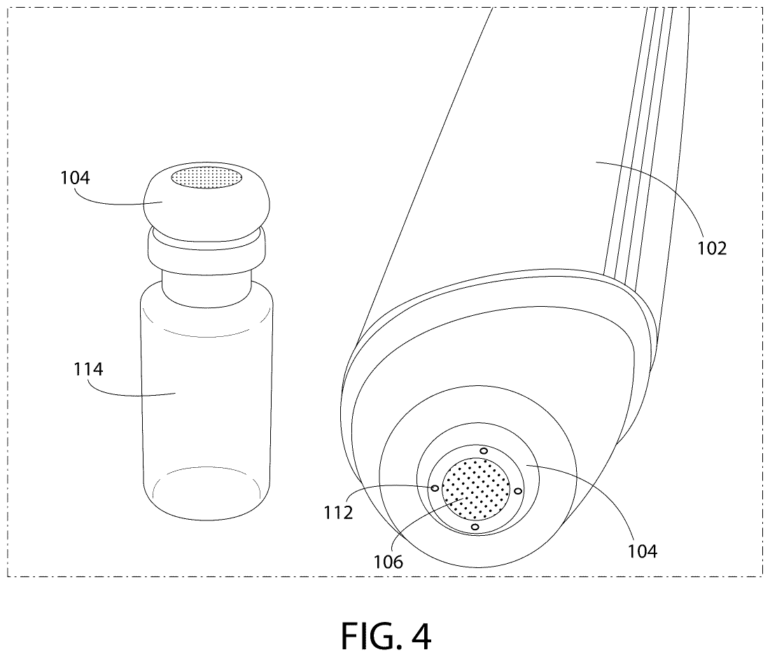

Referring to FIG. 4, the device 100 includes a cartridge 114 configured to house topical serum for delivery during treatment procedures. The cartridge 114 serves as a reservoir that holds serum containing therapeutic compounds to be delivered to the subject's skin. The cartridge 114 is positioned within the body 102 of the device 100, where the hollow internal structure of the body 102 accommodates the cartridge 114 and associated serum delivery components.

As shown in FIG. 4, the cartridge 114 is configured as a separate component that interfaces with the device 100 through insertion into the body 102. The cartridge 114 is designed to fit securely within the internal cavity of the body 102 while maintaining proper alignment with serum delivery pathways and other operational components of the device 100. The positioning of the cartridge 114 within the body 102 enables controlled serum flow from the reservoir to the treatment interface at the head 104.

The cartridge 114 is replaceable, allowing for multiple treatment sessions with fresh serum supplies or different therapeutic formulations. The replaceable nature of the cartridge 114 facilitates efficient treatment workflows by enabling rapid cartridge changes between patients or treatment sessions. The replaceable configuration also supports the use of different serum formulations or concentrations for customized treatment protocols based on specific therapeutic requirements.

With continued reference to FIG. 4, the serum delivery system is configured to transport therapeutic serum from the cartridge 114 to the subject's skin through controlled flow pathways. As the device 100 is actuated during treatment procedures, the serum flows from the cartridge 114 to the subject's skin via outlets included in the nanochip 106. The outlets in the nanochip 106 provide controlled dispensing of therapeutic serum onto the pre-conditioned skin surface during the delivery procedure.

In some cases, the serum may flow through the tips 108 to the subject's skin when the tips 108 are configured as hollow structures. The hollow tips 108 serve as direct conduits for serum delivery, enabling precise placement of therapeutic compounds at the skin interface. The flow through hollow tips 108 provides targeted delivery of serum to specific locations within the treatment area, potentially enhancing the precision and effectiveness of therapeutic compound delivery.

The serum flow pathways from the cartridge 114 are designed to maintain consistent flow rates and serum distribution during treatment procedures. The flow control mechanisms enable uniform serum delivery across the treatment area while preventing excessive serum dispensing that could reduce treatment efficiency. The controlled flow characteristics support optimal therapeutic compound delivery through the enhanced permeability pathways created during the pre-conditioning procedure.

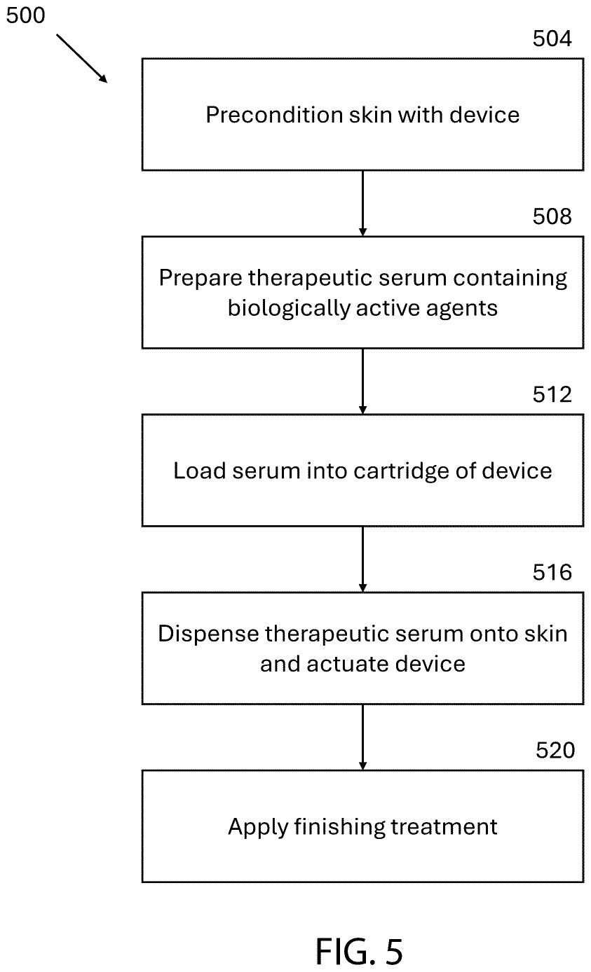

Referring to FIG. 5, a method 500 of performing intradermal delivery using the treatment device 100 will now be described. The method 500 employs a controlled sequence of steps to temporarily disrupt the skin barrier and deliver a therapeutic serum, formulation, or other topical composition into deeper epidermal or dermal layers. Although specific ranges and parameters are provided for the illustrated embodiment, other values and variations may be employed to achieve equivalent results.

In step 504, a pre-conditioning procedure is performed on substantially dry skin using the device 100. The device 100 is applied to a target region of the skin with an empty or non-filled cartridge 114. The head 104 is translated across the target region in one or more motion patterns, such as vertical, horizontal, circular, or diagonal motions, for approximately 15 to 20 seconds per 2×2 cm section area. In alternative embodiments, the duration may range from about 5 seconds to about 60 seconds per 1 to 5 cm² area depending on the desired degree of exfoliation. For example, 10-15 seconds or 20-30 seconds per 5×5 cm area or 4×4 cm area. The mechanical motion may be manual, motorized, or assisted by vibration, and may abrade, exfoliate, or otherwise disrupt the stratum corneum to form transient micro-pathways that increase permeability. The pre-conditioning phase thus prepares the skin surface by locally disrupting the barrier layer without full penetration through the stratum corneum. In certain embodiments, the motion parameters or contact pressure may be automatically regulated by the device 100 to ensure consistent surface preparation.

In step 508, a therapeutic serum, cosmetic formulation, or pharmaceutical composition is prepared for delivery. The formulation may be withdrawn into a syringe, ampule, or other sterile transfer vessel having a volume between about 1 mL and about 5 mL, such as a 3 cc syringe. In some embodiments, the formulation is diluted with a carrier such as saline, sterile water for injection (SWFI), buffered aqueous solution, or other isotonic medium to achieve a desired concentration, viscosity, or osmolarity. The concentration of active components may range from about 0.001 wt. % to about 20 wt. % depending on the intended therapeutic use. The prepared formulation is then transferred into a cartridge 114 or reservoir configured to interface with the delivery device 100.

In step 512, the therapeutic serum is loaded into the cartridge 114, which may include one or more internal chambers or flow control elements. The cartridge 114 is inserted into an instrument inlet of the device 100, optionally engaging with a locking feature, magnetic coupling, or threaded connection. A retaining ring, collar, or other securing element may be installed to hold the cartridge 114 in place. In alternative embodiments, the cartridge may include a self-sealing port, a disposable microchip array, or a replaceable nanotip subassembly configured to maintain sterility and prevent cross-contamination between uses.

In some embodiments, the serum preparation step 208 and the cartridge-loading step 512 may be performed before the pre-conditioning step 504 or concurrently therewith. For example, the formulation may be prepared and loaded in advance to enable immediate use of the device 100 once the skin surface has been pre-conditioned. The relative ordering of these steps may vary depending on workflow preferences, treatment environment, or device configuration, without departing from the scope of the method 500.

In step 516, the device 100 is positioned vertically or at an oblique angle between about 30° and about 90° relative to the skin surface for approximately 3 to 10 seconds to allow proper distribution of the serum within the cartridge. The therapeutic serum is then dispensed through one or more micro-ports 112 located adjacent to the nanochip 106 or other dispensing interface. In the illustrated embodiment, four micro-ports are provided, each having a diameter between about 0.05 mm and about 0.6 mm; however, any number from two to twelve ports may be employed. The device 100 is translated across the pre-conditioned region in vertical, horizontal, or circular patterns for a period ranging from about 1 second to about 120 seconds per treatment zone, thereby enabling uniform infusion of the formulation across the treated surface. In some embodiments, the motion path may be automatically guided by a programmed routine or assisted by feedback sensors configured to monitor contact pressure and coverage.

During serum infusion, the device 100 is actuated to vibrate or oscillate to enhance penetration of the formulation into the epidermal layer. The vibration frequency may range from about 3,000 RPM to about 12,000 RPM, or about 5,800 to about 8,800 RPM in the illustrated embodiment. The vibratory motion may be linear, rotational, orbital, or ultrasonic, and may induce repeated micro-perforations at controlled depths between about 50 μm and about 800 μm. The vibration amplitude may range from about 0.05 mm to about 0.5 mm. These mechanical oscillations promote diffusion and capillary uptake of the formulation through the temporarily disrupted stratum corneum. In alternative embodiments, the vibration may be replaced or supplemented with pneumatic pulsing, micro-needling, or acoustic actuation to achieve comparable dermal penetration.

Upon completion of serum infusion, the treated area may optionally be massaged manually or using the head 104 of the device 100 to promote even distribution and absorption. In some embodiments, the skin is pretreated with a spray to neutralize pH and to help clean or clear skin sebum and oils to effect better absorption. The device 100 may further activate a visible-light source 120 to deliver red, blue, or near-infrared LED therapy either concurrently with or following massage. Red-light wavelengths between about 600 nm and about 700 nm may accelerate tissue healing and reduce inflammation, while blue-light wavelengths between about 400 nm and about 500 nm may provide antimicrobial or soothing benefits. In some embodiments, combined or pulsed light therapy may be applied to achieve synergistic effects with the delivered formulation.

In step 520, one or more finishing treatments such as cooling, moisturizing, or occlusive applications may optionally be applied to complete the protocol. The skin barrier typically recovers within about 5 to 30 minutes, depending on the intensity of treatment, as natural repair processes restore the stratum corneum. This controlled recovery window allows sufficient time for compound uptake while minimizing the duration of barrier compromise. In some embodiments, post-treatment parameters (temperature, moisture level, or impedance) may be monitored to confirm barrier restoration.

The method 500 may be performed as a single treatment or repeated as part of a multi-session regimen. In certain embodiments, the treatment is repeated from about two to about twelve times, such as four to six times, over a period of days or weeks to achieve cumulative or sustained therapeutic effects. The frequency and spacing of sessions may be adjusted based on the subject's response, formulation potency, or intended clinical outcome.

### Examples

The following example illustrates the use of the treatment device 100 and the methodology 500 to demonstrate the controlled disruption and restoration of the skin barrier during intradermal delivery. High-resolution ultrasonic imaging was employed to visualize the effects of the pre-conditioning procedure, compound delivery, and subsequent skin recovery on human scalp tissue.

The imaging results provide experimental validation of the methodology 500, showing that the controlled pre-conditioning phase effectively removes or disrupts the stratum corneum to enhance permeability, that therapeutic compounds penetrate to deeper epidermal and dermal layers during treatment, and that the barrier function is restored within minutes following completion of the procedure.

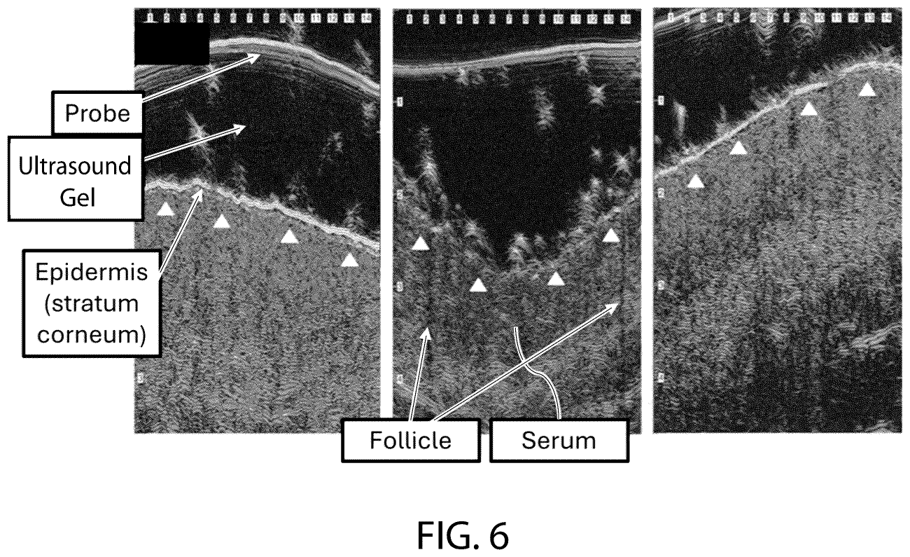

Referring to FIG. 6, the left-most image shows a high resolution ultrasonic view of human scalp prior to any pre-conditioning procedures. The image shows ultrasonic gel separating an ultrasound probe from the subject's skin surface, with white triangular markers indicating the location of the stratum corneum layer. In this pre-treatment condition, the stratum corneum appears as a distinct white line that serves as a physical barrier to the environment. This intact barrier layer demonstrates the natural impediment to topical delivery of therapeutic compounds through conventional application methods.

The stratum corneum barrier shown in the pre-treatment ultrasonic image represents the challenge that conventional topical delivery methods face when attempting to deliver therapeutic compounds to deeper epidermal layers. The intact barrier function of the stratum corneum may prevent or significantly limit the penetration of topically applied therapeutic compounds, resulting in reduced bioavailability and diminished therapeutic effectiveness at the target tissue sites.

The central image in FIG. 6 shows a high resolution ultrasonic view after the scalp has been pre-conditioned with the device 100 and therapeutic compounds have been delivered to the skin. The ultrasonic image demonstrates that the stratum corneum has been removed or substantially removed through the pre-conditioning procedure, thus exposing deeper levels of the epidermis that were previously protected by the intact barrier layer.

As shown in the central ultrasonic image, the previously existing natural barrier has been temporarily eliminated through the mechanical pre-conditioning process. The removal or substantial removal of the stratum corneum creates enhanced permeability pathways that enable the therapeutic serum containing biologically active compounds to permeate through the skin barrier and reach deeper epidermal layers where therapeutic effects may be achieved more effectively.

The central image in FIG. 6 demonstrates that hair follicles serve as conduits to deliver the therapeutic serum to deeper levels of the epidermis and dermis. The ultrasonic imaging shows how the disruption of the stratum corneum around follicular openings enables therapeutic compounds to penetrate along these natural pathways, reaching tissue depths that may not be accessible through conventional topical application methods. The follicular conduits provide additional routes for therapeutic compound delivery beyond the enhanced permeability created through direct stratum corneum disruption.

The therapeutic serum penetration shown in the central ultrasonic image demonstrates the enhanced bioavailability achieved through the pre-conditioning methodology. The therapeutic compounds may reach their intended sites of action in higher concentrations and with greater tissue distribution compared to conventional topical delivery methods that must overcome the intact stratum corneum barrier. This enhanced penetration may translate to improved therapeutic outcomes for various skin conditions and diseases.

The right-most image in FIG. 6 shows a high resolution ultrasonic view taken fifteen minutes after the pre-conditioning procedure was completed. The ultrasonic image demonstrates that the stratum corneum has undergone self-repair and has returned to its normal closed state within this brief timeframe. The restoration of the barrier function shows the temporary nature of the stratum corneum disruption achieved through the pre-conditioning methodology.

The rapid self-repair of the stratum corneum shown in the fifteen-minute post-treatment ultrasonic image demonstrates the skin's continuous and natural healing processes that restore barrier function following the procedure. The quick restoration of the stratum corneum layer may minimize the duration of barrier compromise while providing sufficient time for therapeutic compound penetration during the treatment window. This rapid recovery may reduce the risk of prolonged barrier dysfunction while maintaining the therapeutic benefits achieved during the enhanced permeability phase.

The ultrasonic imaging sequence shown in FIG. 6 provides objective evidence of the controlled and temporary nature of the barrier disruption achieved through the pre-conditioning methodology. The progression from intact barrier function through temporary disruption to restored barrier function demonstrates that the treatment approach may achieve enhanced therapeutic compound delivery without causing permanent damage to the skin's protective mechanisms.

The restoration of normal barrier function within fifteen minutes, as demonstrated in the ultrasonic imaging, may represent an optimal balance between therapeutic compound delivery enhancement and skin safety. The brief window of enhanced permeability may provide sufficient time for therapeutic compound penetration while minimizing exposure to environmental factors that could potentially cause adverse effects through the compromised barrier.

The ultrasonic imaging results shown in FIG. 6 demonstrate the reproducible and predictable nature of the pre-conditioning and delivery methodology. The clear visualization of barrier disruption and subsequent restoration provides evidence that the treatment approach may achieve consistent results across different treatment sessions and subjects. This consistency may support the development of standardized treatment protocols that optimize therapeutic outcomes while maintaining safety parameters.

The clinical demonstration provided by the ultrasonic imaging may validate the theoretical basis for enhanced therapeutic compound delivery through pre-conditioning of the skin barrier. The visual evidence of stratum corneum removal, enhanced compound penetration, and subsequent barrier restoration supports the efficacy of the two-phase treatment approach for improving the bioavailability of topically applied therapeutic compounds in clinical applications.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

1. A method for intradermal delivery of a serum to a subject, comprising:
(a) pre-conditioning a target area of the subject's stratum corneum by contacting the area with a device comprising a cartridge and a nanotip, the pre-conditioning comprising moving the device in vertical, horizontal, and circular motions over the target area for about 15 to 20 seconds per 2×2 cm portion of the area to mechanically abrade the stratum corneum;
(b) filling a cartridge of the device with a serum composition comprising one or more biologically active agents;
(c) inserting the cartridge into an inlet of the device and holding the device vertically for about 5 seconds;
(d) applying the serum to the pre-conditioned area by actuating the device to vibrate at a speed of from about 5800 to about 8800 revolutions per minute (RPM) and dispense the serum through a plurality of micro-ports positioned adjacent the nanotip, the applying comprising moving the device in vertical, horizontal, and circular motions for up to about 60 seconds; and
(e) optionally massaging the serum into the treated area.

2. The method of claim 1, wherein the one or more biologically active agents are selected from the group consisting of peptides, growth factors, platelet-rich plasma, exosomes, secretomes, vitamins, botanicals, minoxidil, finasteride, and mixtures thereof.

3. The method of claim 2, wherein the one or more biologically active agents comprise exosomes.

4. The method of claim 2, wherein the one or more biologically active agents comprise secretomes.

5. The method of claim 1, wherein the serum composition is autologous.

6. The method of claim 1, wherein the serum composition is allogeneic.

7. The method of claim 1, wherein the pre-conditioning in step (a) does not penetrate through the stratum corneum.

8. The method of claim 7, wherein the method is configured to deliver the one or more biologically active agents to at least one of the subject's stratum lucideum, stratum granulosum, stratum spinosum, and stratum basale.

9. The method of claim 1, wherein the target area of the subject's skin is free of the serum composition and biologically active agents prior to performing the pre-conditioning in step (a).

10. The method of claim 1, wherein the nanotip comprises a plurality of tips.

11. The method of claim 10, wherein the plurality of tips are arranged in an array.

12. The method of claim 10, wherein the plurality of tips have a height between 0.12 and 1.0 mm.

13. The method of claim 12, wherein the plurality of tips have a height between 0.12 and 0.3 mm.

14. The method of claim 10, wherein the plurality of tips are made from monocrystalline silicon.

15. The method of claim 1, further comprising a step of applying light from a visible light source to the target area contemporaneously with at least one of the pre-conditioning in step (a) or the applying in step (d).

16. The method of claim 15, wherein the light is red light or blue light.

17. The method of claim 16, wherein the light is red light with a wavelength in the range of 620 and 700 nm.

18. The method of claim 1, wherein the method is repeated 4 to 6 times to achieve optimal therapeutic results.

19. The method of claim 1, wherein the stratum corneum undergoes self-repair and returns to normal barrier function within 10 to 15 minutes after the pre-conditioning.

20. The method of claim 1, wherein the plurality of micro-ports have sizes selected from 0.1 mm, 0.2 mm, and 0.5 mm.
